(19) United States
(12) Patent Application Publication
(10) Pub. No.: US 2004/0122407 A1
(43) Pub. Date: Jun. 24, 2004

(54) Absorbent product

(76) Inventors: Francisco J Valdividia Hernandez, Sao Jse dos Campos (BR); Carlos Da Silva Macedo, Sao Jose dos Campos (BR); Eduardo C Andrea Aledo, Sao Jose dos Campos (BR)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/451,201

(22) PCT Filed: Dec. 18, 2001
(86) PCT No.: PCT/BR01/00153

(30) Foreign Application Priority Data
Dec. 24, 2000 (BR) PI0006236-7

(51) Int. Cl. A61F 13/15

(52) U.S. Cl. 604/385.23; 604/385.08

(57) ABSTRACT

A generally thin, flexible sanitary napkin capable of absorbing medium to high menstrual flows, and having a stiffened center is provided. In one embodiment, the sanitary napkin has a longitudinal central region disposed along the length of at least a portion of the longitudinal centerline, and longitudinal side regions are disposed outboard of the longitudinal central region. The longitudinal central region of the sanitary napkin has a flexure-resistance that is greater than that of the longitudinal side regions. The embodiment of the sanitary napkin described above easily forms around the curvature of the wearer's labia majora by cupping and surrounding the exterior of the labia majora. The longitudinal central region of the sanitary napkin forms the bottom of a cup-like trough under the wearer's labia majora, and the longitudinal side regions are sufficently flexible that they are able to form the sides of the trough structure.

This invention refers to a non-returnable absorbent product, of conforming reaction for use in compressions in the region between the legs through zones of controlled rigidity and the fabrication method thereof.

Examples of the absorbent items that are part of this invention are children's and geriatric diapers as well as diapers against urinary incontinence and non-returnable feminine napkins, of multi-layer type, which include at least one layer permeable to body fluids meant for contact with regions of the body, one impermeable layer on the reverse or external side that remains in contact with the clothes and one central region for body fluid absorption placed between the mentioned layers.

### DESCRIPTION OF THE STATE OF THE ART

Absorbent items of the state of the art reveal several attempts to improve efficiency against leaks, which include variations in the dosage and composition of hydrophilic and hydrophobic materials in the absorbent center and in the layers and various formats meant for an adjustment of the absorbent item to the users' outlines.

In general, the diapers' aspect is that of an hourglass, presenting respective rear and front regions opposed longitudinally and linked by an intermediate region that includes a concentration of absorbent materials and structures, the intermediate region presenting side narrowing for accommodation in the region between the legs and varying the dimensions and forms of the regions according to the manufacturer.

Of a similar conception, hygienic absorbents of the state of the art present narrowing in the intermediate region or also widening articulated in lateral regions. As attempts to resolve and/or improve deficiencies of absorption and retention of fluids in the state of the art absorbents, absorbent cores were created with lateral regions articulated by longitudinal compression lines, extremity regions with hydrophobic compositions, grooves or barrier lines that would confine the fluids within a central region of the absorbent with a higher fluid retention capacity, thus forming an absorption accumulator. Notwithstanding the various combinations of absorbent compositions and formats, the fluid retention capacity also depends on the size and thickness of the absorbent core and on the possibilities of assimilation of the body fluid. On the other hand, the articulation lines or grooves of the absorbent items in the region between the legs have an influence on the accommodation of the absorbent item, subject to the influence of compressions and consequently reflects itself on the efficiency and on the comfort the absorbing item should provide.

In absorbent products of the state of the art, a deformation occurs, due to compression in the region between the legs of the product in use. That deformation occurs due to the pressure exerted by the curvature regions of the legs in movement and of all that region between the user's legs and regarding the user's torso, affecting the performance in use, due to the restrictions created, such as: narrowing, breaks, padding, etc., in the body fluids' impact area. Absorbent products of the state of the art with absorbent cores, rectangular or of formats with narrowing are part of this context.

Also regarding comfort, it is advisable that the absorbent article isn't voluminous, due to the growing use of tighter clothes.

### OBJECTIVES OF THE INVENTION

This invention has, as one of its objectives, the provision of a non-returnable absorbent product, preferably a diaper or a feminine napkin that includes a core of body fluid absorbent with a reaction conformable in use to the relative movement between the internal leg and intimate regions through differentiated rigidity zones that provide better efficiency against leaks, high power of body fluid absorption and utilization comfort.

Another objective is to obtain an absorbent item with the mentioned characteristics and qualities, which is obtained through simple fabrication processes that don't require additional or specific development equipment, thus maintaining the fabrication costs at competitive levels.

### BRIEF DESCRIPTIONS OF THE INVENTION

The above objectives are reached by the absorbent product of this invention, which includes a multi-layer structure that includes an absorbent core and forms the body or chassis, the absorbent core presenting a conformable reaction in compression regions of the Product in use. That conformability of the absorbent product is obtained through rigidity differentials in the core between points of adjacent regions of the core regarding a non-conformable region of the core when submitted to compression during use. The rigidity differentials are obtained through weight differences of the material constituting the absorbent core, by density differentials or increased concentration of that material in that region. Another way of obtaining the rigidity differential is by the superposition of absorbent pieces that provide that rigidity differential.

Areas of the absorbent core with gradual variations are defined as zones with rigidity differentials, in a continuous way, of rigidity in selected directions of the absorbent core, which distinguish the absorbent item of this invention in relation to the state of the art items by the absence of punctual regions with bounds or scaled rigidity creating barriers to the dispersion of the fluid within the absorbing core.

Among the possible absorbent items that are part of an absorbent core pursuant to the teachings of this invention, a diaper with a substantially thin structure and with geometric dimensions and form, of the type of diapers produced with compulsory material loss tending towards zero. Diapers with different chassis formats may also incorporate the core of the characteristics of this invention.

Preferably, the absorbent core is differentially conformable in regions in the front, at the rear and adjacent to the product's compression regions and reaches differentials or incremental variations of rigidity of at least 30%. These conformable portions are deformations responsive to pressures exerted by the circular perimeter of the legs in movement or during the user's accommodation. This rigidity differential can be increasing or decreasing depending on the comfort regions.

As advantages, the invention's core with rigidity differentials can be applied on absorbent items and diapers of little thickness, assuring a directed and differentiated conformation responsive to the product's compression. The result is a continuous adjustment to the body free from tensions, a controlled fluid diffusion and absorption extremely efficient against body fluid leaks, beside providing a comfortable utilization, due to the fact that this construction provides a directed and differentiated conformation of the product, providing adjustment and comfort during the use.

### BRIEF DESCRIPTION OF THE DRAWINGS

This invention shall be, hereinafter, described with more details based on a diaper with one of the possible formats, as in the performance example represented in the drawings of the figures. The figures show:

FIG. 1 is an upper view of a diaper that incorporates the absorbent core of this invention;

FIG. 2 is a side view of the diaper illustrated in FIG. 1;

FIG. 3 is a cross section view of the diaper along the 3-3′ cut line; and

FIG. 4 is a front view of the diaper illustrated in FIGS. 1, 2e 3, when it is in a position of use together with the user's body.

### DETAILED DESCRIPTION OF THE FIGURES

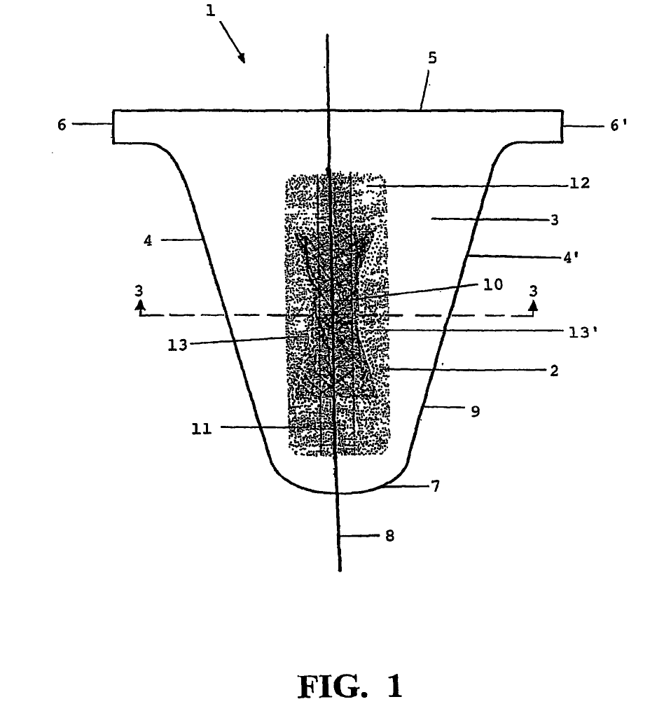

Pursuant to the preferential achievement, FIG. 1 shows a diaper indicated with 1, of the type known by the specialists on the subject as diaper with compulsory material loss tending towards zero. This type of diaper owns a 2 absorbent core disposed in a 3 body or chassis outlined in the transversal direction by a pair of straight and transversally opposed 4.4′ side edges, which converge in a mutual approximation and symmetrically from a 5 rear edge region endowed with 6.6′ fixation extremities for the 7 distal frontal region opposed longitudinally, defining a straight trajectory with a constant inclination angle measured between one of the mentioned 4.4′ side edges and the longitudinal symmetry axis and the 7 symmetry axis of diaper 1.

Body or chassis 3 is formed by a structure that includes a layer permeable to body fluids meant to remain in contact with regions of the body and not showed on the figure, a 9 impermeable layer on the reverse or external side that remains in contact with the clothes and the mentioned 2 core placed between the permeable layer not shown and the 8 impermeable layer mentioned.

Absorbent core 2 has the direction of its longitudinal length substantially coincident with the direction of the longitudinal length of chassis 3, and is placed in a substantially centralized way in relation to the transversal width of chassis 3.

Absorbent core 2 presents a longitudinal region substantially distinct and centralized 10, and substantially frontal longitudinal regions 11, rear 12 or lateral 13.13′ adjacent to portion 10. Portion 10 is centralized between ⅛ and ⅝ of the length of the absorbent product, presenting a rigidity differential of at least 30% from the rigidity presented by the front, rear and lateral adjacent regions. Portion 10 can present the rigidity and conformation differential with curved or diagonal lines in relation to central line 8. Evidently, centralized portion 10 can own a rigidity or smoothness differential superior to portions front 11, rear 12 or lateral 13.13′ adjacent in a proportion of at least 30%, that rigidity difference being selected and determined for convenience of fabrication or utilization, as well as its width and centralization can vary, those variations being merely optional.

As the user's region between the legs normally undergoes compression with deformations that follow the geometry performed by the legs' circular perimeter, the region between the legs is defined as being 10% to 60% of the length of absorbent product 1, from the front part to the rear extremity of absorbent product 1. In this region, absorbent core 2 is placed with differentiated rigidity zones.

The rigidity differential of distinct and centralized portion 10 in relation to front 11, rear 12 and lateral 13.13′ adjacent regions, can be managed via weight differentials of the constituting material the absorbent core, by differentials of density or increased concentration of that material in that portion. Another form of obtainment of the rigidity differential is through superposition of absorbent pieces that provide that rigidity differential. The absorbent pieces can be layers of material, as if they were individual absorbent cores, which are superposed forming a 2 absorbing core, thicker and with higher rigidity in central portion 10.

Variably, a 2 absorbent core can also be conceived, presenting more smoothness in distinct and centralized portion 10. Both possibilities are merely configurative variants, without that positioning causing variations in this invention's protection purpose. Also, analogically to what was commented above, the portion with highest rigidity can be localized in the front, rear or lateral regions adjacent to absorbent core 2, both possibilities being merely configurative variants, without that positioning causing variations in this invention's protection purpose.

In the preferential achievement shown in the figures, especially in FIG. 4, when the user puts on absorbent product 1, front 11, rear 12 and lateral 13.13′ adjacent regions undergo a significant directed deformation due to the pressure/compressing of the region between the legs caused by the circular perimeter of the user's legs, conforming themselves to the latter's body, and distinct and centralized portion 10 remains substantially unaltered, due to its higher rigidity. Such behavior allows the front, rear or lateral adjacent regions to function as “walls”, avoiding the lateral leak of body fluids and leading those fluids to distinct and centralized portion 10, where they will be absorbed.

The regions of absorbent product 1 that present lower rigidity or higher smoothness allow the correct conformation of that absorbent product 1 to the user's body, with comfort and smoothness.

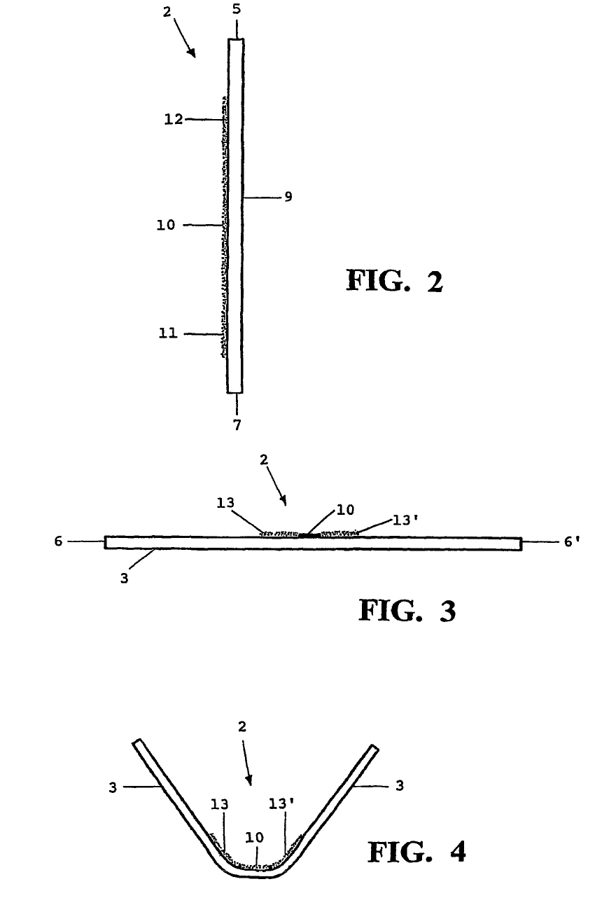

Absorbent product 1 shown in FIGS. 1 to 4 is obtained via a process of production of non-returnable products, which basically includes the stages of:

feeding of a first continuous sheet of flexible impermeable material;

placement of an absorbing core 2;

feeding of a continuous sheet of permeable flexible material;

placement of the fixation and closing systems;

lamination of the components mentioned above; and

cut of the structure.

Preferably, the absorbent core is constituted of traditional absorbent materials, such as cellulose pulp, super-absorbent polymers, textile fibers, cotton, turf moss, sponged materials, among others, agglomerated and with a density between 0.15 g/cm³ and 0.50 g/cm³, with weights between 200 g/m² and 1,500 g/m² and thickness of 0.1 cm to 1.0 cm. The flexibility, measured by the ASTM D4032.82 method, can be of up to 400 g in the area of lowest rigidity and of up to 900 g in the region of highest rigidity, preferably having a relation between the lowest rigidity and highest rigidity regions of 0.30 to 0.44. Auxiliary materials can be added to the structures to facilitate the acquisition and distribution of the body fluids, such as differentiated fabrics with resilient characteristics and hydrophilic treatment. However, again, we are dealing only with configurative possibilities and, possibly, any material that fulfills the objectives defined in the project can be used.

As advantages, the invention presents the fact that it is extremely efficient against body fluid leaks, besides providing a comfortable utilization, due to the fact that this construction provides a directed and differentiated or gradual deformation in the region between the legs, conforming it to the user's body.

A preferred example of achievement having been described, it must be understood that the purpose of this invention covers other possible variations, such as an intimate absorbent item, being limited only by the tenor of the attached claims, including therein the possible equivalents.

1. Absorbent product for personal use of the type that includes at least one layer permeable to body fluids meant to remain in contact with regions of the body, one impermeable layer on the reverse side and one absorbent agent between the mentioned layers, CHARACTERIZED in that the absorbent agent owns rigidity agents of increasing or decreasing differential variations responsive to external compression efforts.

2. Absorbent product, pursuant to claim 1, CHARACTERIZED in that the differential rigidity variations of the mentioned absorbent core are of at least 30% between the core′ regions.

3. Absorbent product, pursuant to 1, CHARACTERIZED in that the absorbent core (2) presents a rigidity differential between a centralized region of the core and adjacent regions of at least 30%.

4. Absorbent product, pursuant to any of claims 1 to 3, CHARACTERIZED in that the rigidity differential is of at least 30%, increasingly or decreasingly.

5. Absorbent product, pursuant to claim 1, CHARACTERIZED in that the rigidity agents of differential variations include absorbent agents of the absorbent core with weights superior or equal to 1,500 g/m².

6. Absorbent product, pursuant to claim 1 or 5, CHARACTERIZED in that the rigidity agents of differential variations include absorbent agents of the absorbent core with a density superior or equal to 0.15 g/cm³ and inferior or equal to 0.50 g/cm³.

7. Absorbent product, pursuant to claim 1, CHARACTERIZED in that the regions with lower rigidity present values of at the maximum 400 grams pursuant to method ASTM D4032.82.

8. Absorbent product, pursuant to claim 1, CHARACTERIZED in that the regions with higher rigidity present values of at the maximum 400 grams pursuant to method.

9. Absorbent product, pursuant to one of claims 1, 7, 8, CHARACTERIZED in that the relation between the regions of lower rigidity and of higher rigidity is of 0.30 to 0.44.

10. Absorbent product, pursuant to claim 1, CHARACTERIZED in that the centralized region of the core presents a rigidity differentiation configuration with curved or diagonal lines in relation to the imaginary central line.

11. Absorbent product, pursuant to claim 1, CHARACTERIZED in that the thickness of the absorbent core is of a value of between 0.1 cm and 1.0 cm.

12. Absorbent product, pursuant to any of the previous claims, CHARACTERIZED in that it includes a diaper.

13. Absorbent product, pursuant to any of claims 1 to 12, CHARACTERIZED in that it includes an intimate absorbent item.

14. Diaper of the type that includes at least one layer permeable to body fluids meant to remain in contact with regions of the body, one impermeable layer on the reverse side and one absorbent core between the mentioned layers, with one rear edge region and one distal frontal region longitudinally opposed to an intermediate region delimited laterally by a pair of straight edges and transversally opposed, defining a straight trajectory with a constant inclination angle measured between one of the mentioned side edges and the diaper's longitudinal symmetry axis, CHARACTERIZED in that it includes the absorbent core such as defined in any of claims 1 to 11.

15. Fabrication process of an absorbent item such as defined in any of the previous claims, CHARACTERIZED by including the stages of
feeding of a first continuous sheet of flexible impermeable material;
placement of an absorbing core 2;
feeding of a continuous sheet of permeable flexible material;
placement of the fixation and closing systems;
lamination of the components mentioned above; and
cut of the structure.
